(19) United States
(12) Patent Application Publication
(10) Pub. No.: US 2020/0214872 A1
(43) Pub. Date: Jul. 9, 2020

(54) A DRAINABLE OSTOMY APPLIANCE

(71) Applicant: SALTS HEALTHCARE LIMITED, Aston, Birmingham, West Midlands (GB)

(72) Inventors: Lee TRETHEWAY, Aston, Birmingham, West Midlands (GB); Lee HOWARD, Aston, Birmingham, West Midlands (GB); Iain POWNER, Aston, Birmingham, West Midlands (GB); Jesus ALFARO, Aston, Birmingham, West Midlands (GB)

(21) Appl. No.: 16/648,321

(22) PCT Filed: Sep. 21, 2018
(86) PCT No.: PCT/GB2018/052693

§ 371 Date: Mar. 18, 2020

(30) Foreign Application Priority Data
Sep. 22, 2017 (GB) 1715401.4

(51) Int. Cl. A61F 5/443; A61F 5/445

(52) U.S. Cl. CPC A61F 5/443; A61F 5/441; A61F 5/445

(57) ABSTRACT

A drainable ostomy appliance including: first and second walls connected to each other at or near their peripheries, the first wall having a stoma-receiving opening; a collecting cavity defined between the first and second walls; a connection member connected to the first wall for attaching the appliance to a user or for attaching the appliance to a flange for attaching the appliance to a user; an outlet which extends away from the stoma-receiving opening, the outlet having an opening for waste and the outlet being formed from at least one wall which terminates at a wall edge; and a stiffening member which is connected to the wall forming the outlet, the stiffening member being positioned such that it overlaps the edge of said wall effectively extending the length of the outlet.

### DESCRIPTION OF INVENTION

The invention relates to ostomy appliances. In particular, but not exclusively, the invention relates to drainable or ileostomy appliances.

It is known to provide drainable ostomy appliances with means to assist in opening and closing an opening thereof to permit the contents of the device to be emptied. However, openings can become stuck in a closed condition, in use. This makes it difficult for a user to manipulate the opening. In particular, if stiffening members are used at or near the exit of the opening and the opening becomes stuck in a closed condition, users may find it difficult to open the opening by applying finger pressure alone to the ends of the stiffening members. This is because the stiffening members will all tend to bow in the same direction, resulting in the opening remaining closed. It can also become difficult to clean the opening of such an ostomy appliance effectively. Ineffective cleaning can result in the leakage of waste from the device, which is clearly undesirable.

The present invention seeks to address these problems.

According to a first aspect of the invention we provide a drainable ostomy appliance including:
first and second walls connected to each other at or near their peripheries, the first wall having a stoma-receiving opening;
a collecting cavity defined between the first and second walls;
a connection member connected to the first wall for attaching the appliance to a user or for attaching the appliance to a flange for attaching the appliance to a user;
an outlet which extends away from the stoma-receiving opening, the outlet having an opening for waste and the outlet being formed from at least one wall which terminates at a wall edge; and
a stiffening member which is connected to the wall forming the outlet, the stiffening member being positioned such that it overlaps the edge of said wall effectively extending the length of the outlet.

Further features of the various aspects of the invention are set out in the clauses which follow the description and the appended claims.

Embodiments of the invention will now be described by way of example only with reference to the accompanying drawings, of which:

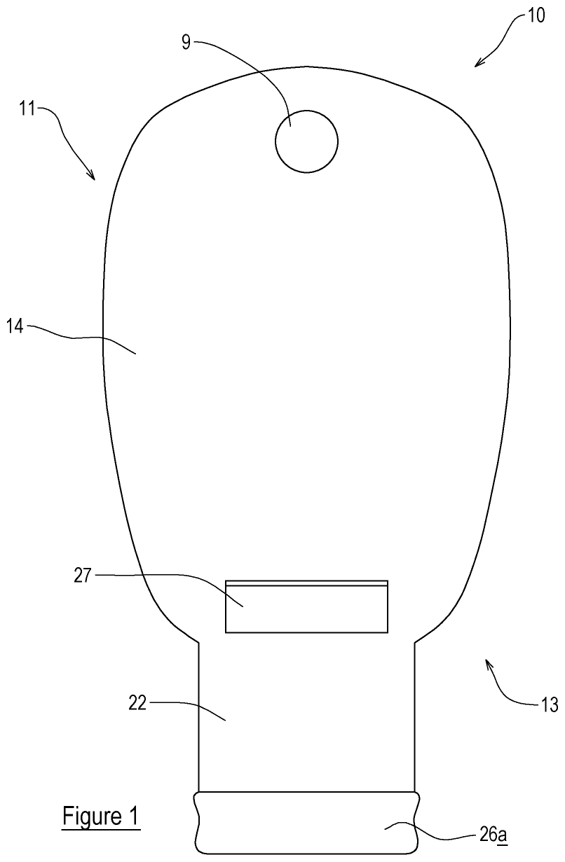

FIG. 1 is a front view of a first embodiment of an ostomy device in accordance with the invention with an outlet thereof shown in an unrolled condition;

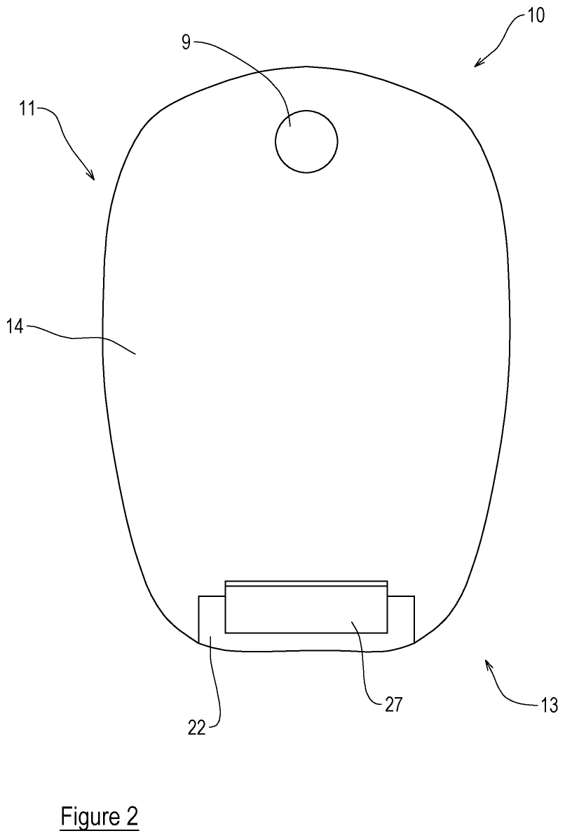

FIG. 2 is a front view of the device of FIG. 1 but with the outlet thereof shown in a rolled, i.e. closed, condition;

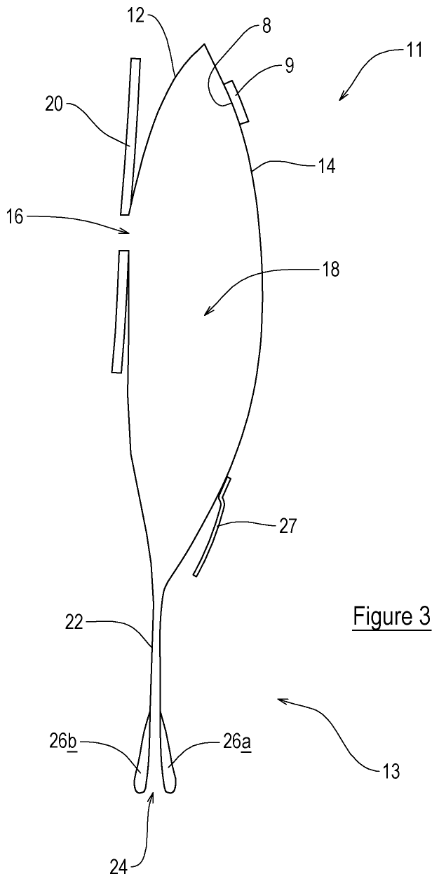

FIG. 3 is a side cross-sectional view of the ostomy device corresponding to FIG. 1;

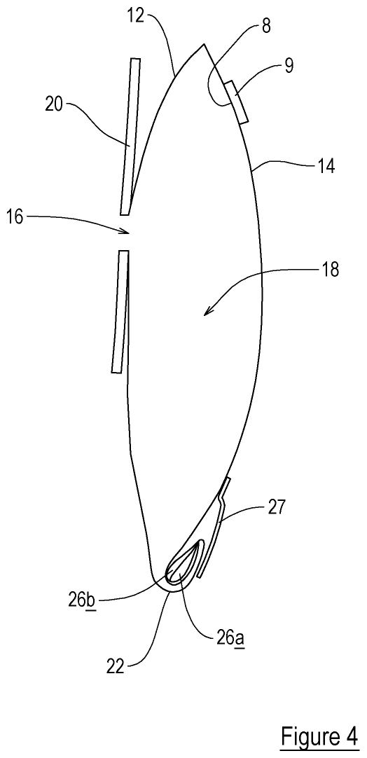

FIG. 4 is a side cross-sectional view of the ostomy device corresponding to FIG. 2;

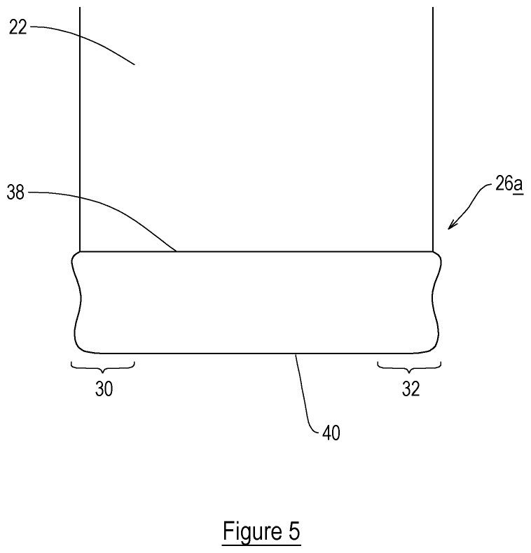

FIG. 5 is an enlarged front view of a stiffening member and outlet of the device of FIG. 1;

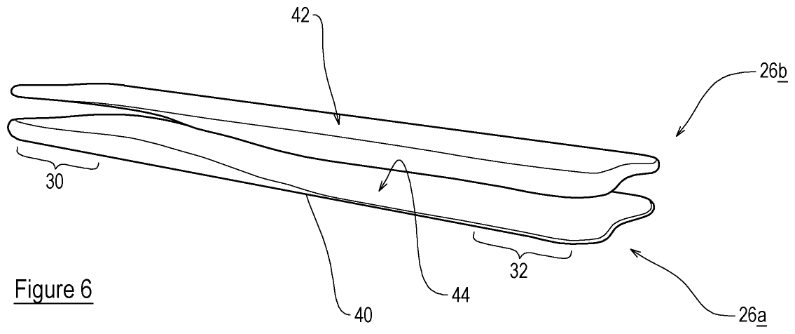

FIG. 6 is a perspective view showing end and side profiles of stiffening members of the ostomy device of FIG. 1;

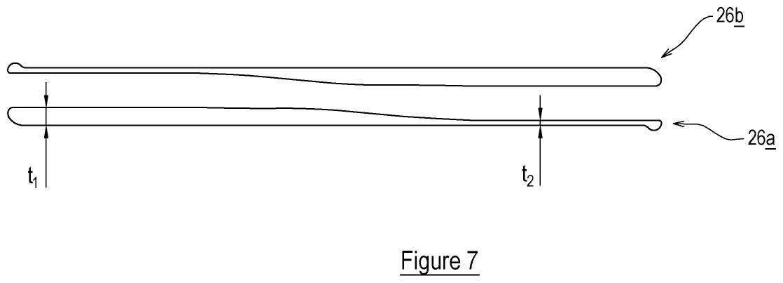

FIG. 7 is an end view showing the end profile of the stiffening members of the ostomy device of FIG. 1;

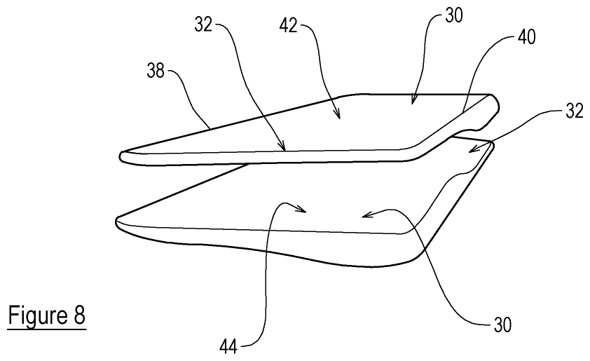

FIG. 8 is a perspective view showing end profiles of the two stiffening members of the ostomy device of FIG. 1;

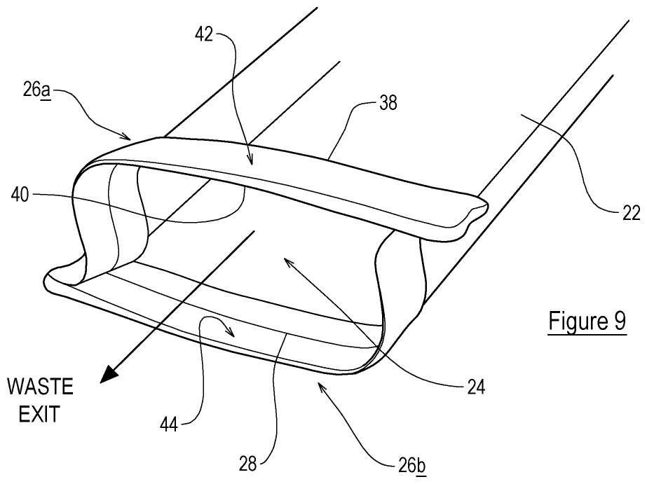

FIG. 9 is a perspective view of the outlet of the ostomy device, showing the separation of the stiffening members from each other during emptying of the device;

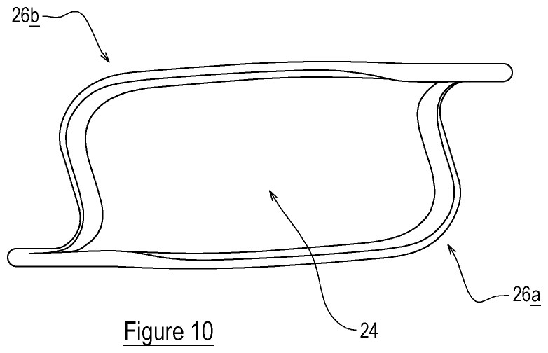

FIG. 10 is an end view (looking into an interior of the ostomy device) of the outlet of the ostomy device, showing the separation of the stiffening members from each other during emptying of the device;

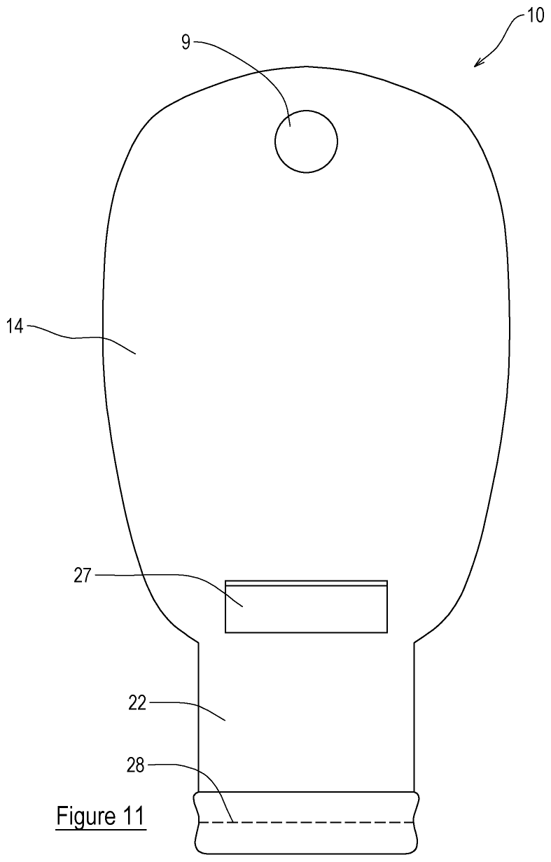

FIG. 11 is a further front view of the ostomy device of FIG. 1 with the outlet shown in an unrolled condition;

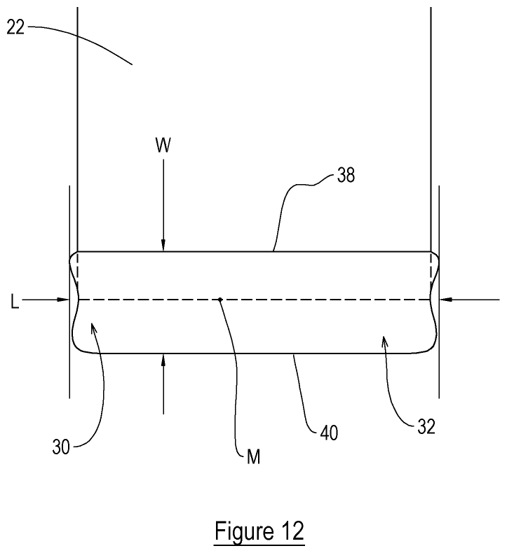

FIG. 12 is a further enlarged front view of the stiffening member and outlet of FIG. 1;

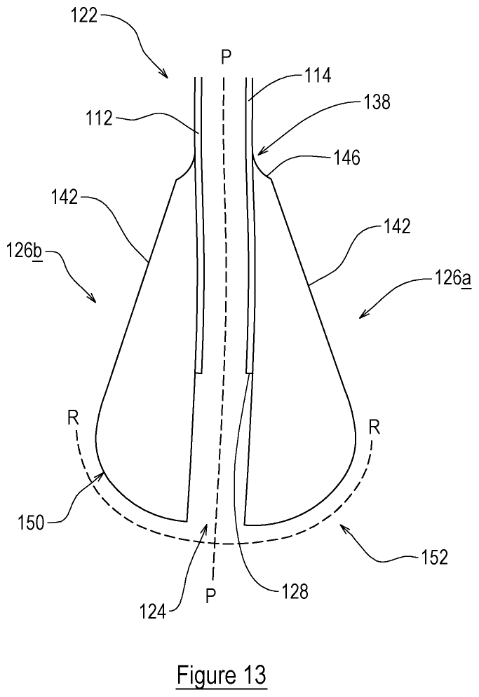

FIG. 13 is a side cross-sectional view of the stiffening members and outlet of a second embodiment of an ostomy device in accordance with the invention, with the outlet thereof shown in an unrolled condition;

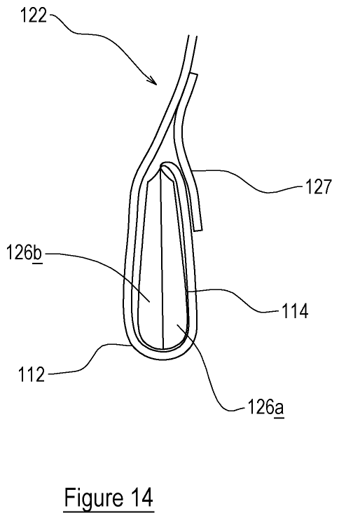

FIG. 14 is a side view of the device of FIG. 13 but with the outlet thereof shown in a rolled, i.e. closed, condition;

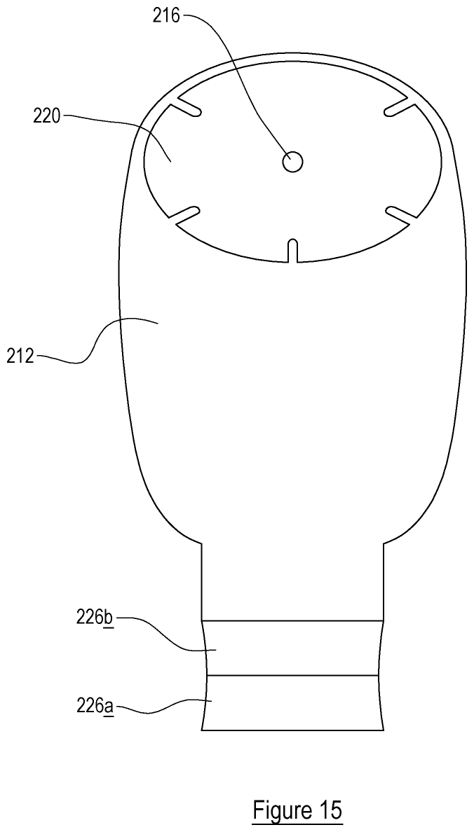

FIG. 15 is a rear view of a third embodiment of an ostomy device in accordance with the invention with an outlet thereof shown in an unrolled condition; and

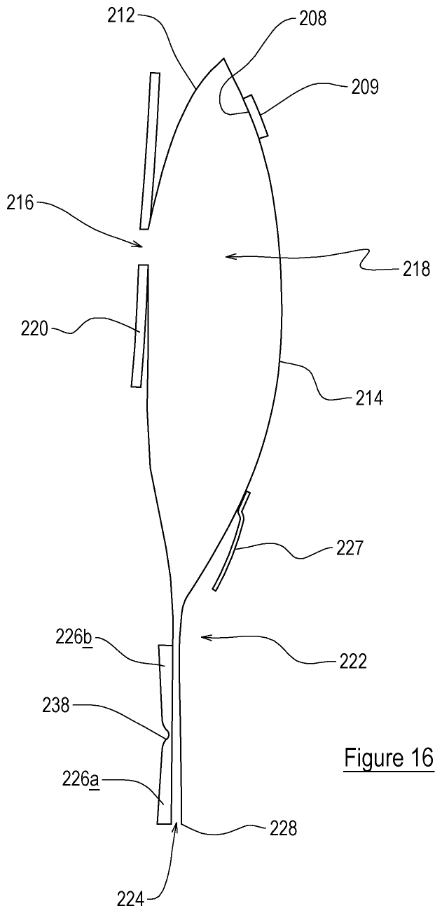

FIG. 16 is a side cross-sectional view of the ostomy device of FIG. 15.

Referring to FIGS. 1 to 12, these show a first embodiment of an ostomy appliance in accordance with the present invention, generally at 10. The ostomy appliance 10 has a top, or upper end, 11 and a bottom, or lower end, 13. The ostomy appliance 10 has first and second walls 12, 14 connected to each other at or near their peripheries, by any suitable means known in the art. For example, they could be adhered to each other or heat welded.

The first wall 12 includes a stoma-receiving opening 16 and supports a connection member or flange 20 for attaching the appliance 10 to a user. In the embodiment shown, the connection member 20 is a hydrocolloid wafer for securing the appliance 10 to the skin of a user around their stoma. Alternatively, the connection member 20 may be for attaching the appliance 10 to a flange for attaching the appliance to a user (e.g. a two-piece appliance as it is known in the art).

Referring to FIG. 3, the first and second walls 12, 14 define a waste collecting cavity 18 therebetween. In some embodiments the first and/or second walls 12, 14 may be covered by comfort layers though these are not essential. The appliance 10 may also have a filter 9 positioned over a suitable gas vent 8 in either or both of the first 12 or second 14 walls, as is well known in the art.

The lower end 13 of the appliance 10 is provided with an outlet 22 formed by portions of the first and second walls 12, 14, again, as is well known in the art. The outlet 22 extends downwardly away from the stoma-receiving opening 16 and terminates at an opening 24. The opening 24 permits waste collected in the collecting cavity 18 to be expelled from the appliance 10.

In the present embodiment first and second elongate stiffening members 26a, 26b are provided to assist with opening and closing of the opening 24. The stiffening members 26a, 26b are connected, e.g. adhered or welded, to the exterior surfaces of the first and second walls 12, 14, respectively, near the opening 24, such that they are positioned one on top of the other (when viewed from the front), e.g. they are positioned generally opposite each other. Both stiffening members extend laterally across the outlet 22 such that they extend generally perpendicularly to the direction in which waste exits the appliance 10, as shown in FIGS. 1 and 3.

The stiffening members 26a, 26b need only be stiff in the sense that they are relatively stiff compared to the walls 12, 14 of the ostomy appliance 10. In this embodiment the stiffening members 26a, 26b are injection moulded from a plastics material.

The outlet 22 may be stored, or stowed, by being rolled up around the stiffening members 26a, 26b, as is generally known in the art, and as shown in FIGS. 2 and 4. Such rolling up uses the laterally extending edges of the members 26a, 26b as a fulcrum(s) to assist in tight closing of the outlet 24, to prevent leaking. In the present embodiment, the stiffening members 26a, 26b are rolled up two or three times, to seal the opening 24 in a closed condition. A flap 27 may be provided to secure the rolled up outlet 22 in its stowed condition. Any suitable fastening means, e.g. hook, and loop (Velcro) or adhesive, may be provided on respective faces of the flap 27 and outlet 22 to secure the outlet 22.

The stiffening members 26a, 26b are substantially identical to each other and are configured such that when one member 26a is rotated 180 degrees and positioned on top of the other member 26b, they mate and cooperate with one another. Thus, the facing surfaces of the members 26a, 26b cooperate with each other so as to substantially close the outlet 22 therebetween. As they are identical, only the shape of the stiffening member 26a will be discussed herein.

When looking at the member 26a from FIG. 5 the member 26a has first and second lateral end portions 30, 32 positioned at either side of the opening 24. First and second laterally extending edges or sides 38, 40 extend across the outlet 22 between the first and second end portions 30, 32, respectively. The first side 38 is positioned closer to the stoma-receiving opening than the second side 40.

The stiffening member 26a is elongate and generally planar, e.g. flat, on its outer surface 42 which faces away from the outlet 22, as shown in FIGS. 6, 7 and 8. However, a surface 44 of the member 26a which faces inwardly towards the outlet 22 (and towards the other member 26b) is contoured, or non-planar. This important shaping is described in more detail below.

As shown in FIGS. 6 to 10, a thickness of the stiffening member varies both between the first and second ends, or end portions, 30, 32 and between the first and second sides 38, 40. It should be noted that FIGS. 6 to 8 show the stiffening members 26a, 26b not connected to their respective walls 12, 14 and with the stiffening members 26a, 26b spaced apart from each other along their entire length. This has been done to aid clarity and to show the contours of the inner, facing, surfaces of the members 26a, 26b. It should be noted that once the stiffening members 26a, 26b are connected to the walls 12, 14, the lateral ends thereof are essentially connected to each other, with the walls 12, 14 sandwiched therebetween, as show in FIGS. 9 and 10.

The thickness of the stiffening member 26a at its first end portion 30 positioned closest to the opening is greater than the thickness of the stiffening member 26a at its second end portion 32 positioned closest to the opening. By this we mean that a thickness of the stiffening member 26a decreases or reduces as it extends from the first end portion 20 to the second end portion 32. In the present embodiment, the reduction is a tapering of the thickness laterally from one side of the stiffening member 26a to the other, but it should also be noted that the degree of taper changes across the stiffening member 26a. FIG. 8 highlights this point, in that the thickness of the end portion 30 increases as it extends from the edge 38 to the edge 40, but the thickness of the end portion 32 is substantially constant from the edge 38 to the edge 40.

It follows therefore that the thickness of the edge 38 of each stiffening member 26a, 26b is substantially constant across the outlet. However, the thickness of the edge 40 of each stiffening member changes as that edge extends from one side of the stiffening member 26a, 26b to the other side of the stiffening member 26a, 26b (as is shown in FIG. 7). This change is represented on the figures by t₁ which is the thickness near the end 30 and t₂ which is the thickness near the end 32. The thickness of the edge 38 is also t₂.

Referring to FIGS. 11 to 12, the outlet 22 is formed from extensions or continuations of the first and second walls 12, 14 which terminate at a wall edge 28. In alternative embodiments the outlet 22 may be formed from only one continuous wall. Indeed, the outlet 22 may be formed separately from the remainder of the ostomy appliance 10 and welded or adhered to the remainder of the appliance 10 when it is manufactured.

Only a portion of each stiffening member 26a, 26b is connected to its respective first or second wall 12, 14. In other words, the stiffening members 26a, 26b are positioned such that they overlap the edges 28 of the first and second walls 12, 14 effectively extending the length of the outlet 22. In other words, the stiffening members 26a, 26b themselves at least partially define the opening 24.

As shown in FIG. 12, the stiffening member 26a has a width W which extends between its first and second sides 38, 40. The stiffening member 26a also has a length L which extends between its first and second ends 30, 32. The stiffening member 26a is positioned such that up to 50% of the width W of the stiffening member 26a at the mid-point M (the mid-point being positioned halfway between the first and second ends 30, 32) extends over the edge 28. Other embodiments have been envisaged where either stiffening member 26a, 26b is positioned such that up to 10%, or up to 20%, or up to 30%, or up to 40%, or up to 50% of the width W of the stiffening member 26a, 26b at the mid-point M extends over the edge 28.

The wall edge 28 is substantially linear and extends generally perpendicularly across the outlet 22. The wall edge 28 may in some embodiments be curved or have a curved portion, e.g. it could be convex or concave.

To open the opening 24 of the appliance 10 the stiffening members 26a, 26b are bent away from each other by applying finger pressure to their first and second respective end portions 30, 32, such that they bow away from each other, as shown in FIGS. 9 and 10. Once finger pressure is removed, the opening 24 moves to or substantially moves to its closed condition due to the stiffening members 26 being biased to a relatively flat position.

When the opening 24 is in a closed condition the inwardly facing surfaces 44 of the first and second stiffening members 26a, 26b cooperate with each other. In doing so, the outwardly facing surfaces 42 of the first and second stiffening members 26a, 26b are either parallel with each other or inclined at an angle to each other. In the present embodiment they are inclined to each other, i.e. the distance between the exterior surfaces increases as the surfaces extend from the edge 38 to the edge 40.

The stiffening members 26a, 26b can be bowed away from each other more easily compared to the prior art. This is because the stiffening members 26a, 26b bow more easily at their relatively thinner portions. This is beneficial because stiffening members can become stuck together, in use, and so by being designed to bow away from each other more easily the stiffening members 26a, 26b are able to more easily bow apart.

FIGS. 13 and 14 show the outlet of a second embodiment of an ostomy appliance in accordance with the present invention. Features which are in common with the first embodiment have been given the same reference numeral with the addition of 100. Those features will not be discussed again here.

The outwardly facing surfaces 142 of the first and second stiffening members 126a, 126b are generally planar, much like in the first embodiment. In this embodiment, as shown in FIG. 13, the inwardly facing surface 144 of each stiffening member 126a, 126b is substantially planar. In addition, the cross-sectional shape, e.g. profile, of each stiffening member 126a, 126b is substantially constant as it extends across the outlet 122. Together the first and second stiffening members 126a, 126b substantially form a teardrop shape, and are mirror images of each other about plane P. A first portion 146 of the outwardly facing surface 142 (positioned closest to the edge 138) is concave. This portion 146 connects to the generally planar surface 142 which in turn continues into an edge portion 152 which is positioned close to the opening 124 of the outlet 122. An exterior surface 150 of the edge portion is convex in the sense that it curves inwardly towards the opening 124.

The edge portions 152 abut or are positioned next to each other, when the opening 124 is closed and form a continuous curved profile. This is shown by the curve R on FIG. 13.

The exterior surface 150 of the edge portion 152 substantially prescribes a portion of a cylindrical surface, defined by the curve R. Thus, when the stiffening members 126a, 126b abut, their combined surfaces 150 substantially prescribe a larger portion of a cylindrical surface, e.g. about half of the surface of a cylinder.

The edge portions 152 are advantageous when rolling up the outlet 122 as it enables any waste trapped in the outlet 122 to be pushed out of the outlet against the edge portions 152. This ensures that a good seal is made and that a minimal quantity of waste can escape through the opening 124 when the outlet is rolled up as shown in FIG. 14.

Other alternative embodiments have been envisaged where only the stiffening member 126a or 126b is provided. In those embodiments the edge portion 152 of the stiffening member may separately form a continuous curved profile which follows a curve similar to R.

FIGS. 15 and 16 show a third embodiment of an ostomy appliance in accordance with the present invention. Features which are in common with the first embodiment have been given the same reference numeral with the addition of 200. Those features will not be discussed again here.

In the third embodiment the first and second stiffening members 226a, 226b are both provided on the first wall 212 (the wall which faces the user) of the outlet 222. The second stiffening member 226b is positioned above the first stiffening member 226a further away from the opening 224 of the outlet 222 than the first stiffening member 226a. In particular, the second stiffening member 226b abuts, or lies close to, the first side 238 of the first stiffening member 226a. The stiffening member 226a tapers, or narrows, as it extends upwardly away from the opening 222 whilst the opposite is true for the stiffening member 226b. The stiffening member 226b tapers, or narrows, in cross section as it extends downwardly towards the stiffening member 226a.

Preferably, no stiffening members are provided on the second wall 214 of the outlet 222. Therefore, the wall edge 228, or at least a portion of the wall edge 228, partially defines the opening 224 together with the lowermost edge of the stiffening member 226a. In this embodiment the surfaces 254 of the edge portions 252 do not follow a continuous curved profile, but they could be of a similar shape to the profile of the stiffening members in the second embodiment.

When it is desired to close the outlet, the first stiffening member 226a is rolled up until folds on to the wall 214, such that the stiffening members 226a, 226b are then positioned opposite each other. Both stiffening members 226a, 226b are then rolled up together, either one or more times and held in place by a flap similar to the flap 27 of the first embodiment.

In other alternative embodiments only a single stiffening member, for example only the first stiffening member 226a or the second stiffening member 226b, may be provided.

When used in this specification and claims, the terms “comprises” and “comprising” and variations thereof mean that the specified features, steps or integers are included. The terms are not to be interpreted to exclude the presence of other features, steps or components.

The features disclosed in the foregoing description, or the following claims, or the accompanying drawings, expressed in their specific forms or in terms of a means for performing the disclosed function, or a method or process for attaining the disclosed result, as appropriate, may, separately, or in any combination of such features, be utilised for realising the invention in diverse forms thereof.

Further features of one or more aspects of the invention are set out in the numbered clauses provided below.

### Clauses

1. A drainable ostomy appliance including:
first and second walls connected to each other at or near their peripheries, the first wall having a stoma-receiving opening;
a collecting cavity defined between the first and second walls;
a connection member connected to the first wall for attaching the appliance to a user or for attaching the appliance to a flange for attaching the appliance to a user;
an outlet which extends away from the stoma-receiving opening, the outlet terminating at an opening; and
a stiffening member positioned at or near the opening which extends across the outlet, the stiffening member having first and second end portions positioned at respective sides of the outlet;
wherein a thickness of a portion of the stiffening member at or near its first end portion is greater than a thickness of a portion of the stiffening member at or near its second end portion.

2. A drainable ostomy appliance according to clause 1 wherein the stiffening member includes a third portion which extends between the first and second end portions.

3. A drainable ostomy appliance according to clause 2 wherein the third portion has a thickness greater than the first end portion.

4. A drainable ostomy appliance according to clause 2 wherein the third portion has a thickness less than the first end portion.

5. A drainable ostomy appliance according to any one of clauses 2 to 4 wherein the third portion has a thickness greater than the second end portion.

6. A drainable ostomy appliance according to any one of clauses 2 to 4 wherein the third portion has a thickness less than the second end portion.

7. A drainable ostomy appliance according to any one of clauses 2 to 6 wherein the thickness of the first end portion tapers as it extends towards the third portion.

8. A drainable ostomy appliance according to any one of clauses 2 to 7 wherein the thickness of the second end portion tapers as it extends towards the third portion.

9. A drainable ostomy appliance according to any preceding clause wherein the stiffening member includes first and second sides which extend across the outlet between the first and second end portions, respectively, the first side being closer to the stoma receiving opening than the second side, wherein a portion (fourth) of the stiffening member positioned at or near the first side, which portion extends generally across the outlet, has a generally uniform thickness.

10. A drainable ostomy appliance according to clause 9 wherein the fourth portion has a thickness less than the thickness of the first end portion.

11. A drainable ostomy appliance according to clause 9 wherein the fourth portion has a thickness greater than the thickness of the first end portion.

12. A drainable ostomy appliance according to any one of clauses 9 to 11 wherein the fourth portion has a thickness less than the thickness of the second end portion.

13. A drainable ostomy appliance according to any one of clauses 9 to 11 wherein the fourth portion has a thickness greater than the thickness of the second end portion.

14. A drainable ostomy appliance according to any one of clauses 9 to 13 wherein the fourth portion has a thickness less than the thickness of the third portion.

15. A drainable ostomy appliance according to any one of clauses 9 to 13 wherein the fourth portion has a thickness greater than the thickness of the third portion.

16. A drainable ostomy appliance according to any one of clauses 9 to 15 wherein the thickness of the first end portion tapers as it extends towards the fourth portion.

17. A drainable ostomy appliance according to any one of clauses 9 to 16 wherein the thickness of the second end portion tapers as it extends towards the fourth portion.

18. A drainable ostomy appliance according to any one of clauses 9 to 17 wherein the thickness of the third portion tapers as it extends towards the fourth portion.

19. A drainable ostomy appliance according to any preceding clause wherein an outwardly facing surface of the stiffening member, which surface faces away from the outlet, is substantially planar.

20. A drainable ostomy appliance according to any preceding clause including a second stiffening member positioned at or near the opening opposite the (first) stiffening member, said second stiffening member having one or more or all of the features of the first stiffening member as set out in clauses 1 to 19.

21. A drainable ostomy appliance according to clause20 wherein the second stiffening member is complementary to the first stiffening member.

22. A drainable ostomy appliance according to clause 20 or clause 21 wherein inwardly facing surfaces of the first and second stiffening members, which surfaces face towards the outlet, are non-planar and cooperate with each other such that when the opening is in a closed condition outwardly facing surfaces of the first and second stiffening members, which surfaces face away from the outlet, are generally planar and are either parallel with each other or inclined at an angle to each other.

23. A drainable ostomy appliance including:
first and second walls connected to each other at or near their peripheries, the first wall having a stoma-receiving opening;
a collecting cavity defined between the first and second walls;
a connection member connected to the first wall for attaching the appliance to a user or for attaching the appliance to a flange for attaching the appliance to a user;
an outlet which extends away from the stoma-receiving opening, the outlet terminating at an opening; and
a stiffening member positioned at or near the opening which extends across the outlet, the stiffening member having first and second end portions positioned at respective sides of the outlet;
wherein a thickness of the stiffening member decreases or reduces as it extends from its first end portion to its second end portion.

24. A drainable ostomy appliance according to clause 23 including one or more or all of the features as set forth in clauses 2 to 22.

25. A drainable ostomy appliance including:
first and second walls connected to each other at or near their peripheries, the first wall having a stoma-receiving opening;
a collecting cavity defined between the first and second walls;
a connection member connected to the first wall for attaching the appliance to a user or for attaching the appliance to a flange for attaching the appliance to a user;
an outlet which extends away from the stoma-receiving opening, the outlet terminating at an opening; and
a stiffening member positioned at or near the opening which extends across the outlet, the stiffening member having first and second end portions positioned at respective sides of the outlet;
wherein a portion of the stiffening member positioned between its first and second end portions has a thickness which decreases or reduces as it extends from the first end portion to the second end portion.

26. A drainable ostomy appliance according to clause 25 including one or more or all of the features as set forth in clauses 2 to 24.

27. A drainable ostomy appliance including:
first and second walls connected to each other at or near their peripheries, the first wall having a stoma-receiving opening;
a collecting cavity defined between the first and second walls;
a connection member connected to the first wall for attaching the appliance to a user or for attaching the appliance to a flange for attaching the appliance to a user;
an outlet which extends away from the stoma-receiving opening, the outlet terminating at an opening; and
a stiffening member positioned at or near the opening which extends across the outlet, the stiffening member having first and second sides which extend across the outlet;
wherein the first side is closer to the stoma receiving opening than the second side, and
wherein a thickness of the stiffening member increases as it extends from the first side to the second side.

28. A drainable ostomy appliance according to clause 27 wherein a thickness of a portion of the stiffening member between its first and second sides increases linearly as it extends from the first side to the second side.

29. A drainable ostomy appliance according to clause 27 or clause 28 wherein the thickness of the stiffening member tapers as it extends from the second side towards the first side.

30. A drainable ostomy appliance according to any one of clauses 27 to 29 wherein a thickness of the stiffening member at or near the second side is greater than a thickness of the stiffening member at or near the first side.

31. A drainable ostomy appliance according to any one of clauses 27 to 30 wherein a thickness of a portion of the stiffening member positioned between its first and second sides increases as it extends from the second side towards the first side.

32. A drainable ostomy appliance according to clause 30 wherein a thickness of a portion of the stiffening member positioned between its first and second sides increases linearly as it extends from the second side towards the first side.

33. A drainable ostomy appliance according to any one of clauses 27 to 32 wherein an outwardly facing surface of the stiffening member, which surface faces away from the outlet, is substantially planar.

34. A drainable ostomy appliance according to any one of clauses 27 to 33 wherein a portion (first) of an outwardly facing surface of the stiffening member is concave.

35. A drainable ostomy appliance according to clause 34 wherein the concave portion is positioned at or near the first side of the stiffening member.

36. A drainable ostomy appliance according to clause 34 or clause 35 wherein a second portion of an outwardly facing surface of the stiffening member is concave.

37. A drainable ostomy appliance according to clause 36 wherein the second concave portion is positioned at or near the second side of the stiffening member.

38. A drainable ostomy appliance according to any one of clauses 27 to 37 wherein a portion (first) of an outwardly facing surface of the stiffening member is convex.

39. A drainable ostomy appliance according to clause 38 wherein the convex portion is positioned at or near the second side of the stiffening member.

40. A drainable ostomy appliance according to clause 38 or clause 39 wherein a second portion of an outwardly facing surface of the stiffening member is convex.

41. A drainable ostomy appliance according to clause 40 wherein the second convex portion is positioned at or near the first side of the stiffening member.

42. A drainable ostomy appliance according to any one of clauses 27 to 41 wherein an inwardly facing surface of the stiffening member, which surface faces towards the outlet, is substantially planar.

43. A drainable ostomy appliance according to any one of clauses 27 to 42 including a second stiffening member including one or more or all of the features of the first stiffening member as set out in clauses 1 to 16.

44. A drainable ostomy appliance according to clause 43 wherein the second stiffening member is positioned at or near the opening.

45. A drainable ostomy appliance according to clause 44 wherein the second stiffening member is positioned generally opposite the first stiffening member.

46. A drainable ostomy appliance according to clause 43 wherein the second stiffening member is positioned above the first stiffening member, on a same wall which forms the outlet, further from the opening of the outlet than the first stiffening member.

47. A drainable ostomy appliance according to clause 46 wherein the second stiffening member abuts or lies close to the first side of the first stiffening member.

48. A drainable ostomy appliance according to any one of clauses 43 to 47 wherein the second stiffening member is substantially the same as the first stiffening member.

49. A drainable ostomy appliance including:
first and second walls connected to each other at or near their peripheries, the first wall having a stoma-receiving opening;
a collecting cavity defined between the first and second walls;
a connection member connected to the first wall for attaching the appliance to a user or for attaching the appliance to a flange for attaching the appliance to a user;
an outlet which extends away from the stoma-receiving opening, the outlet having an opening for waste and the outlet being formed from at least one wall which terminates at a wall edge; and
a stiffening member which is connected to the wall forming the outlet, the stiffening member being positioned such that it overlaps the edge of said wall effectively extending the length of the outlet.

50. A drainable ostomy appliance according to clause 49 wherein at least a portion of the wall edge partially defines the opening.

51. A drainable ostomy appliance according to clause 49 or clause 50 wherein the stiffening member has first and second sides, which extend across the outlet, and first and second ends which extend between the first and second sides; wherein the first side is closer to the stoma receiving opening than the second side.

52. A drainable ostomy appliance according to clause 51 wherein the stiffening member is positioned such that 10% of a width of the stiffening member extending between the first and second sides, at a mid-point between the first and second ends, extends over the edge of the at least one wall.

53. A drainable ostomy appliance according to clause 51 wherein the stiffening member is positioned such that 20% of a width of the stiffening member extending between the first and second sides, at a mid-point between the first and second ends, extends over the edge of the at least one wall.

54. A drainable ostomy appliance according to clause 51 wherein the stiffening member is positioned such that 30% of a width of the stiffening member extending between the first and second sides, at a mid-point between the first and second ends, extends over the edge of the at least one wall.

55. A drainable ostomy appliance according to clause 51 wherein the stiffening member is positioned such that 40% of a width of the stiffening member extending between the first and second sides, at a mid-point between the first and second ends, extends over the edge of the at least one wall.

56. A drainable ostomy appliance according to clause 51 wherein the stiffening member is positioned such that 50% of a width of the stiffening member extending between the first and second sides, at a mid-point between the first and second ends, extends over the edge of the at least one wall.

57. A drainable ostomy appliance according to any one of clauses 52 to 56 wherein the mid-point is half way between the first and second ends.

58. A drainable ostomy appliance according to any one of clauses 49 to 57 wherein the outlet is formed by an extension or continuation of the first and second walls which define the waste collection cavity.

59. A drainable ostomy appliance according to any one of clauses 49 to 58 including a second stiffening member, including one or more or all of the features of the first stiffening member as set forth in clauses 49 to 57.

60. A drainable ostomy appliance according to clause 59 when dependent on clause 58 wherein the first and second stiffening members are positioned on the first wall and wherein the second stiffening member is positioned above the first stiffening member further from the opening of the outlet than the first stiffening member.

61. A drainable ostomy appliance according to clause 60 wherein the second stiffening member abuts or lies close to the first side of the first stiffening member.

62. A drainable ostomy appliance according to clause 59 wherein the second stiffening member is positioned generally opposite the first stiffening member.

63. A drainable ostomy appliance according to any one of clauses 59 to 62 wherein the second stiffening member is substantially the same as the first stiffening member.

64. A drainable ostomy appliance including:
first and second walls connected to each other at or near their peripheries, the first wall having a stoma-receiving opening;
a collecting cavity defined between the first and second walls;
a connection member connected to the first wall for attaching the appliance to a user or for attaching the appliance to a flange for attaching the appliance to a user;
an outlet which extends away from the stoma-receiving opening, the outlet terminating at an opening;
a first stiffening member positioned at or near the opening and extending across the outlet, the first stiffening member having an edge portion which is positioned close to the opening of the outlet;
a second stiffening member positioned at or near the opening and extending across the outlet, the second stiffening member having an edge portion which is positioned close to the opening of the outlet;
wherein the edge portions abut or are positioned next to each other when the opening is closed and wherein the edge portions form a continuous curved profile.

65. A drainable ostomy appliance according to clause 64 wherein an outwardly facing surface of the first stiffening member, which faces away from the outlet, and an inwardly facing surface of the first stiffening member, which faces towards the outlet, are connected by a surface of the edge portion of the first stiffening member which extends away from the opening and which is convex with respect to a plane which extends generally perpendicular to a plane in which the first and/or second stiffening members generally lie.

66. A drainable ostomy appliance according to clause 64 or clause 65 wherein an outwardly facing surface of the first stiffening member, which faces away from the outlet, and an inwardly facing surface of the first stiffening member, which faces towards the outlet, are connected by a surface of the edge portion of the first stiffening member which extends away from the opening and which is concave with respect to a plane which extends generally perpendicular to a plane in which the first and/or second stiffening members generally lie.

67. A drainable ostomy appliance according to any one of clauses 64 to 66 wherein an outwardly facing surface of the first stiffening member, which faces away from the outlet, and an inwardly facing surface of the first stiffening member, which faces towards the outlet, are connected by a surface of the edge portion which prescribes at least a portion of a cylindrical surface.

68. A drainable ostomy appliance according to any one of clauses 64 to 67 wherein an outwardly facing surface of the first stiffening member which faces away from the outlet is substantially planar.

69. A drainable ostomy appliance according to any one of clauses 64 to 68 wherein a portion of an outwardly facing surface of the first stiffening member which faces away from the outlet is concave.

70. A drainable ostomy appliance according to any one of clauses 64 to 69 wherein the second stiffening member has one or more or all of the features as set forth in clauses 65 to 69.

71. A drainable ostomy appliance according to any one of clauses 64 to 70 wherein the first and second stiffening members have first and second sides which extend across the outlet, where the first side is closer to the stoma-receiving opening than the second side, and wherein the edge portions of the first and second stiffening members are generally symmetrical about a plane which extends in between the first and second stiffening members.

72. A drainable ostomy appliance according to any one of clauses 64 to 71 wherein the first and second stiffening members have first and second sides which extend across the outlet, where the first side is closer to the stoma-receiving opening than the second side, and wherein the first and second stiffening members are generally symmetrical about a plane which extends in between the first and second stiffening members.

1. A drainable ostomy appliance including:
first and second walls connected to each other at or near their peripheries, the first wall having a stoma-receiving opening;
a collecting cavity defined between the first and second walls;
a connection member connected to the first wall for attaching the appliance to a user or for attaching the appliance to a flange for attaching the appliance to a user;
an outlet which extends away from the stoma-receiving opening, the outlet having an opening for waste and the outlet being formed from at least one wall which terminates at a wall edge; and
a stiffening member which is connected to the wall forming the outlet, the stiffening member being positioned such that it overlaps the edge of said wall effectively extending the length of the outlet.

2. A drainable ostomy appliance according to claim 1 wherein at least a portion of the wall edge partially defines the opening.

3. A drainable ostomy appliance according to claim 1 wherein the stiffening member has first and second sides, which extend across the outlet, and first and second ends which extend between the first and second sides; wherein the first side is closer to the stoma receiving opening than the second side.

4. A drainable ostomy appliance according to claim 3 wherein the stiffening member is positioned such that 10% of a width of the stiffening member extending between the first and second sides, at a mid-point between the first and second ends, extends over the edge of the at least one wall.

5. A drainable ostomy appliance according to claim 3 wherein the stiffening member is positioned such that 20% of a width of the stiffening member extending between the first and second sides, at a mid-point between the first and second ends, extends over the edge of the at least one wall.

6. A drainable ostomy appliance according to claim 3 wherein the stiffening member is positioned such that 30% of a width of the stiffening member extending between the first and second sides, at a mid-point between the first and second ends, extends over the edge of the at least one wall.

7. A drainable ostomy appliance according to claim 3 wherein the stiffening member is positioned such that 40% of a width of the stiffening member extending between the first and second sides, at a mid-point between the first and second ends, extends over the edge of the at least one wall.

8. A drainable ostomy appliance according to claim 3 wherein the stiffening member is positioned such that 50% of a width of the stiffening member extending between the first and second sides, at a mid-point between the first and second ends, extends over the edge of the at least one wall.

9. A drainable ostomy appliance according claim 4 wherein the mid-point is half way between the first and second ends.

10. A drainable ostomy appliance according to claim 1 wherein the outlet is formed by an extension or continuation of the first and second walls which define the waste collection cavity.

11. A drainable ostomy appliance according to claim 1 wherein the appliance comprises two stiffening members constituting a first stiffening member and a second stiffening member.

12. A drainable ostomy appliance according to claim 11 wherein the outlet is formed by an extension or continuation of the first and second walls which define the waste collection cavity; and
wherein the first and second stiffening members are positioned on the first wall and wherein the second stiffening member is positioned above the first stiffening member further from the opening of the outlet than the first stiffening member.

13. A drainable ostomy appliance according to claim 12 wherein the second stiffening member abuts or lies close to the first side of the first stiffening member.

14. A drainable ostomy appliance according to claim 11 wherein the second stiffening member is positioned generally opposite the first stiffening member.

15. A drainable ostomy appliance according to claim 11 wherein the second stiffening member is substantially the same as the first stiffening member.

16. (canceled)

17. A drainable ostomy appliance according to claim 12 wherein the second stiffening member is substantially the same as the first stiffening member.

18. A drainable ostomy appliance according to claim 13 wherein the second stiffening member is substantially the same as the first stiffening member.

19. A drainable ostomy appliance according to claim 14 wherein the second stiffening member is substantially the same as the first stiffening member.
